(19) United States
(12) Patent Application Publication
(10) Pub. No.: US 2019/0192701 A1
(43) Pub. Date: Jun. 27, 2019

(54) METHOD FOR TUMOR-TARGETING TREATMENT AND DIAGNOSIS, CONJUGATES USED FOR THE SAME, AND PREPARATION METHOD THEREOF

(71) Applicant: Korea Institute of Radiological & Medical Sciences, Seoul (KR)

(72) Inventors: Tae Sup Lee, Wonju-si (KR); Ik Joon Choi, Seongnam-si (KR); Joo Hyun Kang, Seoul (KR)

(73) Assignee: Korea Institute of Radiological & Medical Sciences, Seoul (KR)

(21) Appl. No.: 16/232,538

(22) Filed: Dec. 26, 2018

(30) Foreign Application Priority Data
Dec. 27, 2017 (KR) 10-2017-0181513

(51) Int. Cl. A61K 51/10; C07K 16/28; A61P 35/00

(52) U.S. Cl. CPC A61K 51/103; A61P 35/00; C07K 16/2863

(57) ABSTRACT

Provided are a conjugate including an EGFR inhibitor that enables treatment and diagnosis of epidermal growth factor receptor (EGFR)-overexpressing tumors including resistant tumors, a method that enables drug delivery treatment and diagnosis of EGFR-overexpressing tumors using the conjugate, and a method of preparing the conjugate.

### CROSS-REFERENCE TO RELATED APPLICATION

This application claims the benefit of Korean Patent Application No. 10-2017-0181513, filed on Dec. 27, 2017, in the Korean Intellectual Property Office, the disclosure of which is incorporated herein in its entirety by reference.

### BACKGROUND

### 1. Field

The present disclosure relates to a theragnosis-based method for tumor-targeting treatment and diagnosis, and more particularly, a method that enables simultaneous treatment and diagnosis of tumors caused by overexpression of epidermal growth factor receptor (EGFR), conjugates used in the method, and a method of preparing the conjugates.

### 2. Description of the Related Art

Traditional anti-cancer agents attack not only cancer cells but also normal cells to exhibit serious side effects, and therefore, many studies have been conducted on cancer therapy which targets specific molecules based on an understanding of molecular biological mechanisms and signal transduction mechanisms of cancer development, namely, molecular targeted therapy. Further, studies have been continued on theragnosis technologies that enable simultaneous diagnosis and treatment to provide personalized medical care that adjusts the type and dose of a drug, taking into account a patient's individual characteristics and disease conditions.

Head and neck cancer is a representative cancer that occurs in the oral cavity, esophagus, and larynx, and is the seventh most common cancer, with an annual worldwide incidence rate of about 600,000. In particular, Korean patients with head and neck cancer are gradually increasing by 2,500 per year, from 12,500 patients in 2002 to 25,000 patients in 2007. More than half of the patients are diagnosed at a locally advanced stage, and in the past decades, most of them have been treated with a combination of surgery, radiation, and chemotherapy. Surgical resection is a primary treatment modality for head and neck cancer. In the case of a locally advanced disease, a combined therapy of chemotherapy and radiotherapy following surgical resection is used as a secondary treatment modality. Further, when recurrence or distant metastasis occurs, various types of chemotherapy (Cisplatin, 5-FU, etc.) and radiotherapy are used in combination, but this provides no change in the survival rate. More than half of patients with head and neck cancer die within 5 years, and patients with recurrence or distant metastasis die within one year. That is, survival rates are very low. Over 80-100% of head and neck cancers are generally epidermal growth factor receptor (EGFR)-positive tumors. Therefore, among EGFR-targeting therapies, immunotherapy with cetuximab is performed, but most cases show no response due to resistance. Accordingly, there is an urgent need for new therapeutic agents. Although there are many studies on EGFR-targeting, drugs that have been developed until now have shown efficacy on very few patients or no satisfactory therapeutic efficacy.

Patent Document 1 discloses a cancer cell-specific drug delivery method using a liposome which is surface-modified with a cell membrane fixation compound containing a protein binding to a target specifically expressed on the surface of cancer cells overexpressing EGFR. However, since this liposome is an artificial membrane made of lipid components, it is recognized as foreign matter introduced into the body and is easily destroyed or eliminated by hydrolysis. Therefore, there are disadvantages in that its effectiveness is difficult to accurately predict, its application to lipophilic drugs is restricted, and in-vivo diagnosis is difficult. Further, there still remains a need to improve upon the limitations of existing chemotherapies, such as non-specific anti-cancer effects, severe systemic toxicity, and secondary cancer development due to field cancerization.

The present inventors, as a result of continuous research, have developed a conjugate including an EGFR inhibitor as a drug carrier, which enables simultaneous diagnosis and treatment of tumors to realize selection and treatment of a therapeutic target and has high therapeutic efficacy against tumors resistant to existing anticancer agents, a method for treatment and diagnosis by tumor-targeting drug delivery using the conjugate, and a method of preparing the conjugates.

### PRIOR ART DOCUMENTS

[Patent Document]

(Patent Document 1) Korean Patent Publication No. 10-2009-0078512

### SUMMARY

An aspect provides a conjugate including an EGFR inhibitor that enables treatment and diagnosis of epidermal growth factor receptor (EGFR)-overexpressing tumors including resistant tumors.

Another aspect provides a method for drug delivery treatment and diagnosis of EGFR-overexpressing tumors including resistant tumors.

Still another aspect provides an efficient, cost-effective method of preparing the conjugate.

### BRIEF DESCRIPTION OF THE DRAWINGS

These and/or other aspects will become apparent and more readily appreciated from the following description of the embodiments, taken in conjunction with the accompanying drawings in which:

FIG. 1 is a conceptual diagram of ¹⁷⁷Lu-PCTA-cetuximab according to one specific embodiment;

FIGS. 2A and 2B show Micro-SPECT/CT images of ¹⁷⁷Lu-PCTA-cetuximab in SNU-1066 HNSCC xenograft models, in which FIG. 2A shows a SPECT image of tumors unresponsive to cetuximab immunotherapy, and FIG. 2B shows a SPECT image of tumors unresponsive to immunotherapy, which was acquired using Lu-177-PCTA-cetuximab after an injection of excess cetuximab for blocking to evaluate specificity of the image;

FIG. 3 shows radioimmunotherpeutic efficacy of ¹⁷⁷Lu-PCTA-cetuximab in SNU-1066 HNSCC xenograft models (data represents mean relative tumor volumes±SD. *, P<0.05; a.u., arbitrary unit); and

FIGS. 4A and 4B show small animal PET imaging of ⁶⁴Cu-PCTA-cetuximab in SNU-1066 HNSCC xenograft models, in which FIG. 4A shows that ⁶⁴Cu-PCTA-cetuximab was selectively localized in an SNU-1066 tumor, and FIG. 4B shows that in blocking experiments, ⁶⁴Cu-PCTA-cetuximab uptake was reduced by pre-treated cold excess cetuximab (tumors are indicated by white dotted lines. T, SNU-1066 tumor; L, liver).

### DETAILED DESCRIPTION

An aspect provides a conjugate of the following Formula 1:

[X]—R-epidermal growth factor receptor (EGFR) inhibitor [Formula 1]

In Formula 1, X represents a radioisotope and R represents a ligand.

As used herein, the term “conjugate” refers to a compound having a ligand and a biologically active molecule. In one specific embodiment, the conjugate refers to a linkage of a drug or an antibody with another agent, e.g., a ligand including a metal chelate, or e.g., an imaging probe including a radioisotope, or a chemotherapeutic agent, a toxin, an immunotherapeutic agent, etc. The linkage may be a covalent bond or a non-covalent interaction such as those through an electrostatic force. In one specific embodiment, the conjugate may be used as an “antibody drug conjugate” or “immunoconjugate”. For example, when the drug is an antibody drug, the conjugate may be used as an “antibody drug conjugate” or “immunoconjugate”, and in this case, the conjugate refers to a linkage of an antibody or an antigen-binding fragment thereof. Further, various linkers known in the related art may be used to form the conjugate or antibody drug conjugate.

As used herein, the term “epidermal growth factor receptor (EGFR)” refers to a type 1 membrane protein of 170 kDa, which is used interchangeably with an epithelial cell proliferation factor receptor or epidermal cell growth factor receptor, and is a cell surface receptor for extracellular protein ligands of the epidermal growth factor family, a subfamily of four closely related kinases, EGFR, HER2/c-neu, Her 3, and Her 4. Activation of the receptor is known to be very important for the innate immune response in epidermal cells and known to be overexpressed in various kinds of tumors.

For example, overexpression of EGFR is observed in cancers of lung cancer, breast cancer, colon cancer, stomach cancer, brain cancer, bladder cancer, head cancer, neck cancer, ovarian cancer, and prostate cancer. Tumor cells in which EGFR is overexpressed may produce epidermal growth factor (EGF) and transforming growth factor-α (TGF-α) which are ligands of EGFR. In this regard, the ligands bind to EGFR to induce cell proliferation and tumor growth. Therefore, when binding of EGF to EGFR is inhibited using an antibody against EGFR as an EGFR inhibitor, growth of cancer cells may be suppressed to treat cancer.

Further, since EGFR mRNA and protein are overexpressed in about 40˜90% of head and neck cancer, EGFR targeted therapy may become a good solution for head and neck cancer.

In one specific embodiment, the EGFR inhibitor may be a drug containing an antibody or an antigen-binding fragment thereof. As used herein, the term “antibody” may also include antibody fragments produced by modification of whole antibodies, antibody fragments synthesized de novo using recombinant DNA methods [e.g., single chain Fv (scFv), single variable domain (Dab)], or antibody fragments confirmed using display libraries, e.g., phage, E. coli, or yeast display libraries. In one specific embodiment, the EGFR inhibitor may be a monoclonal antibody.

In one specific embodiment, the EGFR inhibitor may be any one selected from the group consisting of cetuximab, panitumumab, gefitinib, erlotinib, icotinib, lapatinib, neratinib, apatinib, ceritinib, dacomitinib, and canertinib. In one specific embodiment, the EGFR inhibitor may be cetuximab.

The cetuximab is a monoclonal antibody which is a drug binding to EGFR on the cell surface to block important pathways that promote cell proliferation, thereby inhibiting proliferation of cancer cells, and is commercially available under the trade name Erbitux™, etc. This drug is mainly used in colorectal cancer and head and neck cancer, and also used in various other diseases.

As used herein, the term “ligand” refers to an arbitrary molecule or compound that specifically or selectively interacts with or binds to a second molecule or compound. In one specific embodiment, the ligand may be an antibody or an antigen-binding fragment thereof. For example, the ligand may be an aptamer, a peptide (e.g., peptibody) that specifically interacts with a particular antigen, a receptor molecule, and an antigen-binding scaffold.

In one specific embodiment, the ligand (R) may be a metal chelate ligand that is conjugated to the EGFR inhibitor and chelates a radioisotope to form a complex.

In one specific embodiment, a radioisotope (X) may coordinate with the ligand. As used herein, “ligand→X” means a complex formed by a coordinate bond of the radioisotope (X) with the ligand.

In one specific embodiment, the ligand (R) may be any one selected from the group consisting of 3,6,9,15-tetraazabicyclo[9.3.1 ]-pentadeca-1(15),11,13-triene-3,6,9,-triacetic acid (PCTA), 1,4,7,10-tetraazacyclododecane-1,4,7,10-tetraacetic acid (DOTA), diethylene triamine penta acetic acid (DTPA), 1,4,7,10-tetraazacyclododecane-1,4,7-triacetic acid (DO3A), 1,4,7-triazacyclononane-1,4,7-triacetic acid (NOTA), 1,4,7-triazacyclononane, 1-glutaric acid-4,7-acetic acid (NODAGA), 1,4,8,11-tetraazacyclotetradecane-N,N′,N″,N′″-tetraacetic acid (TETA), 1,4,8,11-tetraazacyclotetradecane-1,4,8-triacetic acid (TE3A), 1,4,8,11-tetraaza bicyclo hexadecane-4,11-diacetic acid (TE2A), cyclen, cyclam, and desferrioxamine (DFO or DFOA). In one specific embodiment, the ligand (R) may be PCTA. The PCTA is useful in that it is more stable at room temperature and less accumulated in the human body.

In one specific embodiment, the conjugate may be a conjugate of the following Formula 2, wherein the EGFR inhibitor is cetuximab:

cetuximab-R→[X] [Formula 2]

In Formula 2, R represents a ligand, X represents a radioisotope, “ligand→X represents a complex formed by a coordinate bond of the radioisotope (X) with the ligand, and “cetuximab-R→[X]” represents a conjugate wherein the ligand is linked to cetuximab by a covalent bond or a non-covalent interaction such as an electrostatic force, and the radioisotope coordinates with the ligand to form a complex.

In one specific embodiment, the ligand may be a metal chelate which may be conjugated to cetuximab. For example, the ligand may be PCTA. In one specific embodiment, the conjugate may be ¹⁷⁷Lu-PCTA-cetuximab. In another specific embodiment, the conjugate may be ⁶⁴Cu-PCTA-cetuximab. In still another specific embodiment, the conjugate may be ⁶⁴Cu/¹⁷⁷Lu-PCTA-cetuximab.

The PCTA-conjugated cetuximab antibody enables effective imaging diagnosis of EGFR-expressing tumors by an imaging device, for example, single photon emission computed tomography (SPECT), and at the same time, treatment of tumors using beta rays emitted from labeled ¹⁷⁷Lu capable of killing cells. Accordingly, effective treatment of tumors unresponsive to cetuximab immunotherapy is possible.

As used herein, the term “radioisotope (X)” refers to an isotope having radioactivity among isotopes of an element. These radioisotopes have different modes of decay depending on the species, and release radiation having unique energy and decay into stable isotopes. The mode of decay includes α decay, β-decay, β+ decay, as well as EC decay in which a K orbital electron is captured by the nucleus. Most of these isotopes release extra energy and become stable isotopes. A quantity of the radioisotope is expressed as a radiation intensity, i.e., the number of disintegration per unit time, and 1 curie (1 Ci) is equal to 3.7×10¹⁰ disintegration/sec (dps).

Since the radioisotopes emit radiation easily detectable even if they exist in a very small amount, the radioisotopes may be used to trace movement of substances. Further, since the radioisotopes give physical or chemical actions to substances during irradiation of the radioisotopes, and have different properties of transmission and scattering depending on substances, they may be employed in basic or applied researches due to these properties, and also in clinical diagnosis or treatment of cancer. Further, radiopharmaceuticals which are prepared using the radioisotopes for simultaneous diagnosis and treatment of diseases are called convergence radiopharmaceuticals.

In one specific embodiment, the radioisotope(X) may be any one selected from the group consisting of lutetium-177 (¹⁷⁷Lu), copper-64 (⁶⁴Cu), copper-67 (⁶⁷Cu), gallium-68 (⁶⁸Ga), zirconium-89 (⁸⁹Zr), yttrium-86 (⁸⁶Y), yttrium-90 (⁹⁰Y), technetium-99m (⁹⁹mTc), indium-111 (¹¹¹In), promethium-149 (¹⁴⁹Pm), scandium-47 (⁴⁷Sc), rhodium-105 (¹⁰⁵Rh), tin-117m1 (^{117m1}Sn), tin-117m2 (^{117m2}Sn), terbium-161 (¹⁶¹Tb), and a combination thereof. In one specific embodiment, the radioisotope(X) may be lutetium-177 (¹⁷⁷Lu), copper-64 (⁶⁴Cu), or a combination thereof.

The lutetium-177 (¹⁷⁷Lu) is a beta ray-emitting radioisotope easily produced in a nuclear reactor, and has a high beta energy of 0.5 MeV or more, and is uniformly irradiated within an irradiation range of 1 mm to 10 mm in tumor to destroy a lesion tissue. The ¹⁷⁷Lu also emits gamma rays of 0.2 MeV, which allows easy diagnosis, as compared with a pure beta-emitting isotope, and has a half-life of 6 days to 7 days (6.65˜6.73 day). Since the ¹⁷⁷Lu emits beta rays as well as gamma rays, therapeutic purposes and nuclear medicine imaging may be obtained at the same time. Further, the ¹⁷⁷Lu is advantageous in that it is applicable to small tumors, because of its cost-effectiveness and a short penetration distance.

The copper-64 (⁶⁴Cu) is a radioisotope produced and purified by irradiating a nickel target with protons accelerated by a cyclotron (particle accelerator), and has a half-life of 12 hours (12.7 hours). The ⁶⁴Cu emits beta rays as well as positrons, and therefore, is suitable for use in convergence radiopharmaceuticals.

In one specific embodiment, the EGFR inhibitor is cetuximab, the radioisotope (X) is lutetium-177 (¹⁷⁷Lu) or copper-64 (⁶⁴Cu), and the ligand (R) may be a metal chelate ligand that may form a complex with [¹⁷⁷Lu] or [⁶⁴Cu] and may be conjugated to cetuximab.

In one specific embodiment, the conjugate may detect a spatiotemporal position by an imaging device while having therapeutic functions, and therefore, a noninvasive, quantitative tumor diagnosis is possible. In one specific embodiment, the conjugate may be imaged by any one selected from the group consisting of positron emission tomography (PET), single-photon emission computed tomography (SPECT), magnetic resonance imaging (MRI), computed tomography (CT), an optical imaging technique, an ultrasound (US) technique, Cherenkov luminescence imaging, and a combination thereof. In one specific embodiment, localization of the conjugate may be detected by SPECT using gamma rays, which allows tumor diagnosis. In another specific embodiment, tumor size and position may be diagnosed by PET or immuno-PET imaging.

In one specific embodiment, the conjugate may be used to efficiently evaluate the localization by the imaging device, which allows selection of a therapeutic target, as well as treatment of tumors unresponsive to existing chemotherapy, for example, immunotherapy of an antibody drug acting as an EGFR inhibitor, e.g., cetuximab. Particularly, the conjugate may be efficiently used for the treatment of head and neck cancer.

Another aspect provides a method for tumor treatment and diagnosis by targeted drug delivery, the method including using the EGFR inhibitor, ligand (R), and radioisotope (X). In the present disclosure, the method for simultaneous treatment and diagnosis by the drug delivery may be referred to as theranostics or theragnosis. Further, in the present disclosure, theranostics or theragnosis refers to a technology that enables simultaneous diagnosis and therapy, and refers to a personalized medical technology capable of adjusting the type and dose of a drug taking into account patient's individual characteristics and disease conditions, and furthermore, simultaneously treating and diagnosing a disease.

In one specific embodiment, the method may be a method for tumor treatment and diagnosis by targeted drug delivery, wherein the conjugate of the following Formula 1 is used:

[X]-R-EGFR inhibitor [Formula 1]

(wherein X is a radioisotope and R is a ligand).

The method, based on theranostics or theragnosis, may realize simultaneous tumor-targeting treatment and diagnosis.

In one specific embodiment, the tumor may be head and neck cancer. In one specific embodiment, the tumor may be an EGFR inhibitor-resistant tumor. In one specific embodiment, the tumor may be a tumor unresponsive to immunotherapy even if an antibody drug used as an EGFR inhibitor is treated thereto. In another specific embodiment, the tumor may be head and neck cancer resistant to EGFR inhibitors.

Response to chemotherapy may be divided into four categories as shown in the following Table 1 according to changes in the lesion size (European society of radiology, Poster No.: C-2149, Congress: ECR 2010, Response assessment in oncology: Past, present and future).

TABLE 1

| | |
|---|---|
| Complete | Disappearance of all target lesions with no evidence of |
| Response (CR) | tumor |
| Partial | At least 30% decrease in the total tumor measurement |
| response (PR) | confirmed |
| | (a repeat observation on two occasions 4 weeks apart) |
| Progressive | At least 20% increase in the total tumor measurement, |
| disease (PD) | taking as reference the smallest sum on study |
| | including baseline, or appearance of one or more new |
| | lesions |
| Stable disease | Fulfilled the criteria for neither PR nor PD |
| (SD) | |

In Table 1, the cases of PD and SD, except CR and PR, are considered to be clinically resistant to the therapy.

The method for treatment and diagnosis according to one specific embodiment also exhibits therapeutic effects on tumors resistant to clinical therapy using EGFR inhibitors. In one specific embodiment, the tumor may be head and neck cancer resistant to a clinical therapy using cetuximab.

In one specific embodiment, the EGFR inhibitor may be cetuximab and the tumor may be a tumor resistant to cetuximab.

In one specific embodiment, the ligand (R) may be 3,6,9,15-tetraazabicyclo[9.3.1]-pentadeca-1(15),11,13-triene-3,6,9,-triacetic acid (PCTA).

In one specific embodiment, the radioisotope (X) may be lutetium-177(¹⁷⁷Lu), copper-64(⁶⁴Cu), or a combination thereof.

The method for treatment and diagnosis according to one specific embodiment may be used in the treatment of cancer caused by overexpression of EGFR. The cancer caused by overexpression of EGFR may include head cancer, neck cancer, lung cancer, breast cancer, colon cancer, stomach cancer, brain cancer, bladder cancer, ovarian cancer, prostate cancer, colorectal cancer, etc. In one specific embodiment, the cancer may be head and neck cancer or head and neck squamous cell carcinoma (HNSCC). In one specific embodiment, the cancer may be cancer resistant to anti-cancer agents.

The method for treatment and diagnosis according to one specific embodiment may exhibit diagnostic and therapeutic effects on tumors even by administration of a single dose of cetuximab antibody, which is not expected to show therapeutic effects, e.g., a single low-dose (100 μg of antibody dose/mouse).

Still another aspect provides a method of preparing the conjugate which is composed of ([X]-R-EGFR), the method including conjugating the ligand (R) to the EGFR inhibitor; and labeling the EGFR inhibitor-conjugated ligand (R) with the radioisotope (X).

In one specific embodiment, the labeling may be performed by forming a complex by coordinating the radioisotope (X) with the ligand (R).

In the radiolabeling, the reaction mixture may be a buffer solution of acetate, e.g., sodium acetate. In one specific embodiment, the buffer solution may be allowed to react at a concentration of about 500 mM to about 1,000 mM. In one specific embodiment, the radiolabeling of ⁶⁴Cu may be performed at room temperature (about 15° C. to about 25° C.). In another specific embodiment, the radiolabeling of ¹⁷⁷Lu may be performed at about 35° C. to about 40° C., for example, at about 37° C.

In one specific embodiment, ⁶⁴Cu may be produced by 50 MeV cyclotron irradiation. The ⁶⁴Cu or ¹⁷⁷Lu may be a commercially available product.

In one specific embodiment, ⁶⁴CuCl₂ (74 MBq) or ¹⁷⁷LuCl₃ (74˜740 MBq) may be added to 1 mg of PCTA-cetuximab. In one specific embodiment, the reaction mixture may be incubated under shaking at room temperature or 37° C. for 1 hour.

In one specific embodiment, radiolabeling yield and purity may be assessed by instant thin-layer chromatography silica gel (Pall Corp.) as a stationary phase and 20 mM citrate buffer (pH 5) containing 50 mM EDTA as a mobile phase. Radiochemical purity may also be confirmed by size-exclusion high-performance liquid chromatography.

As used herein, the term “about” or “approximately” means that a mentioned value may vary to some degree. For example, the value may vary to 10%, 5%, 2%, or 1%. In a specific embodiment, the value may vary to 5%, 2%, or 1%. For example, “about 5” means including any value between 4.5 and 5.5, between 4.75 and 5.25, or between 4.9 and 5.1, or between 4.95 and 5.05.

As used herein, the term “have”, “may have”, “include”, or “may include” indicates existence of corresponding features (e.g., elements such as numeric values or components) but do not exclude presence of additional features. Further, the singular forms include plural referents unless the context clearly dictates otherwise. Unless otherwise defined herein, all technical or scientific terms used herein may have the same meaning that is generally understood by a person skilled in the art. If there is contradiction, adjustment will be made in the present disclosure.

A conjugate according to an aspect enables treatment and diagnosis of EGFR-overexpressing tumors, is effective for tumors that are resistant to a single EGFR inhibitor therapy, and efficiently determines localization by an imaging device, which allows selection of a therapeutic target as well as treatment of tumors resistant to immunotherapy of an existing antibody drug acting as an EGFR inhibitor, e.g., cetuximab.

A method for treatment and diagnosis according to another aspect enables simultaneous treatment and diagnosis of EGFR-overexpressing tumors, and enables effective drug delivery treatment and non-invasive diagnosis of tumors that are resistant to or less responsive to a single EGFR inhibitor therapy.

A preparation method according to still another aspect provides an efficient, cost-effective method of preparing the conjugate according to an aspect.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

Hereinafter, the present disclosure will be described in more detail with reference to the following Examples. However, the following Examples are for illustrative purposes only, and the scope of the present disclosure is not intended to be limited thereto.

### PREPARATION EXAMPLE 1

### Preparation of ¹⁷⁷Lu-PCTA-Cetuximab

(1) Preparation of PCTA-cetuximab Conjugate

Cetuximab (20 mg) was reacted with a bi-functional chelator, p-SCN-Bn-PCTA (Macrocyclics, Dallas, Tex., USA) (10 equivalents) in 100 mM sodium bicarbonate buffer at pH 8.5 and room temperature for 2 hours, and left at 4° C. overnight. Thereafter, unconjugated chelator was removed by dialysis. The immunoconjugate was finally concentrated to 2 mg/mL in 20 mM sodium acetate buffer of pH 6.5. To determine the number of chelates per antibody, mass spectrometry was performed using MALDI mass spectrometry (Voyager-DE STR, PerSpective Biosystems Inc., KBSI, Ohchang, Republic of Korea).

(2) Radiolabeling

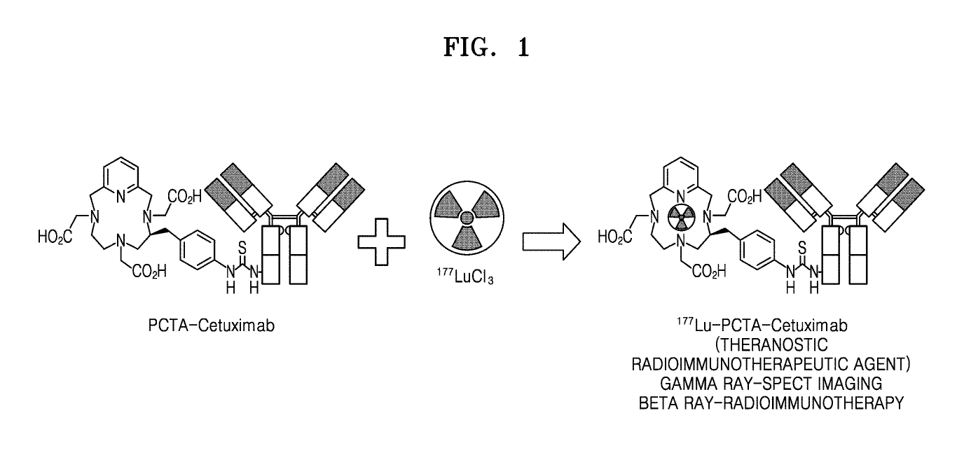

A radioisotope ¹⁷⁷Lu was purchased from ITM AG. ¹⁷⁷LuCl₃ (74˜740 MBq) was added to 1 mg of PCTA-cetuximab. This reaction mixture was incubated at room temperature or 37° C. for 1 hour with constant shaking. The reaction mixture was reacted in a buffer solution of 500 mM to 1,000 mM sodium acetate at 37° C. Radiolabeling yield and purity were assessed by instant thin-layer chromatography silica gel (Pall Corp.) as a stationary phase and 20 mM citrate buffer of pH 5 with 50 mM EDTA. Radiochemical purity was also confirmed by size-exclusion high-performance liquid chromatography. FIG. 1 is a conceptual diagram of ¹⁷⁷Lu-PCTA-cetuximab according to one specific embodiment.

### PREPARATION EXAMPLE 2

### Preparation of ⁶⁴Cu-PCTA-Cetuximab

A radioisotope ⁶⁴Cu was produced by 50 MeV cyclotron irradiation at KIRAMS. ⁶⁴Cu-PCTA-cetuximab was prepared in the same manner as in Preparation Example 1, except that ⁶⁴CuCl₂ (74 MBq) was added to 1 mg of PCTA-cetuximab.

### EXAMPLE 1

### Micro-SPECT/CT Imaging of ¹⁷⁷Lu-PCTA-Cetuximab

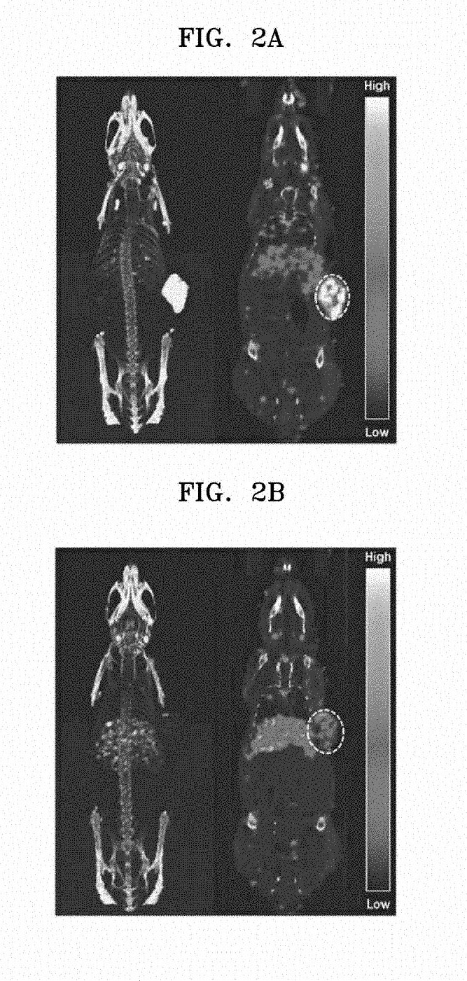

Micro-SPECT/CT imaging was performed to investigate in vivo behavior of ¹⁷⁷Lu-PCTA-cetuximab. Relative volume images and coronal section images of SNU-1066 tumor-bearing mice at 7 day after injection of ¹⁷⁷Lu-PCTA-cetuximab (12.95 MBq, 100 μg) are shown in FIG. 2A and FIG. 2B. Micro-SPECT/CT imaging was performed using a NanoSPECT/CT tomograph (Bioscan, Poway, Calif., USA) for 120 min acquisition. Cone-beam CT images were acquired (180 projections, 1 s/projection, 45 kVp, 177 μA) before micro-SPECT imaging. Co-registration of micro-SPECT and CT images was performed using InVivoScope software (ver. 2.0, Bioscan).

FIGS. 2A and 2B show Micro-SPECT/CT images of ¹⁷⁷Lu-PCTA-cetuximab in SNU-1066 HNSCC xenograft models.

FIG. 2A shows a SPECT image of tumors unresponsive to cetuximab immunotherapy. Specifically, FIG. 2A shows SPECT/CT volume and coronal section images acquired for 2 hours with small animal SPECT/CT system (Bioscan) at 7 days post-injection of ¹⁷⁷Lu-PCTA-cetuximab (12.95 MBq/100 μg). ¹⁷⁷Lu-PCTA-cetuximab was selectively localized in SNU-1066 tumors and showed relatively low uptake in the liver.

FIG. 2B shows a SPECT image of tumors unresponsive to immunotherapy, which was acquired using Lu-177-PCTA-cetuximab after an injection of excess cetuximab (2 mg/head) for blocking to evaluate specificity of the image. Specifically, FIG. 2B shows SPECT/CT volume and coronal section images which were acquired by 2 hr pre-injection of excess cetuximab before administration of ¹⁷⁷Lu-PCTA-cetuximab. In blocking experiments, the uptake of ¹⁷⁷Lu-PCTA-cetuximab was significantly reduced by treatment of excess dose of cold Cetuximab, indicating that ¹⁷⁷Lu-PCTA-cetuximab was localized in EGFR-expressing SNU-1066 HNSCC xenograft models.

### EXAMPLE 2

### Measurement of Efficacy of Radioimmunotherapy

In vivo radiotherapeutic efficacy of ¹⁷⁷Lu-PCTA-cetuximab in SNU-1066 tumor models was investigated. First, SNU-1066 cells were injected into the right flank of mice. When tumor volume reached 100 mm³ to 200 mm³, radioimmunotherapy with ¹⁷⁷Lu-PCTA-cetuximab was performed. SNU-1066 tumor-bearing mice were randomly divided into three groups (n=6 or 7 per group). HNSCC tumor mice were intravenously administrated with saline (control), cetuximab (5 mg/kg, single dose) or ¹⁷⁷Lu-PCTA-cetuximab (12.95 MBq, single dose, 5 mg/kg), respectively. Tumor volume and body weight were measured for 30 day post-treatment.

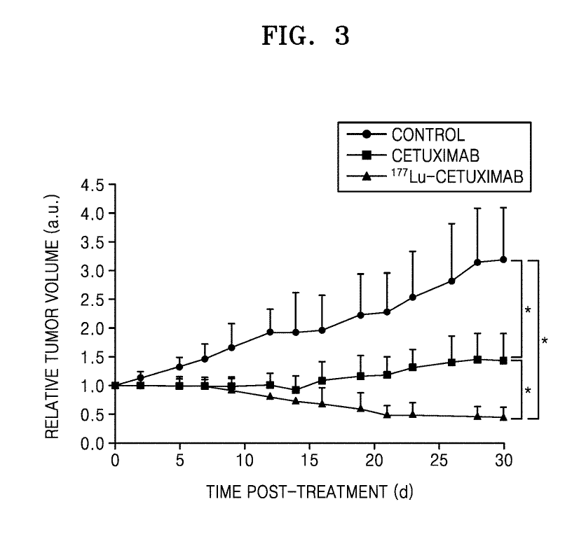

FIG. 3 shows radioimmunotherapeutic efficacy of ¹⁷⁷Lu-PCTA-cetuximab in SNU-1066 HNSCC xenograft models. Data represents mean relative tumor volumes±SD. *, P<0.05; a.u., arbitrary unit.

The treatment was initiated on day 0. SNU-1066 tumor-bearing mice were treated with saline, cetuximab (single dose), and ¹⁷⁷Lu-PCTA-cetuximab (12.95 MBq/100 μg). Tumor volume was calculated by caliper measurement. A time-dependent increase in tumor volume was observed in the saline-treated group. Cetuximab-treated group maintained the growth of SNU-1066 tumors for 2 weeks. A single dose of cetuximab treatment slightly delayed or inhibited the tumor growth during treatment. However, tumor volume re-increased, and tumor regrowth was observed in cetuximab-treated groups and the average tumor volume increased until 30 day. In contrast, a single-dose injection (12.95 MBq) of ¹⁷⁷Lu-PCTA-cetuximab showed marked regression of tumor volume. The tumor volume in ¹⁷⁷Lu-PCTA-cetuximab-treated group on day 30 showed a 55% reduction, as compared with tumor volume before treatment. The tumor volume in ¹⁷⁷Lu-PCTA-cetuximab-treated group showed a statistically significant difference, as compared with that in saline- and single dose of cetuximab-treated groups (P<0.05). SNU-1066 tumor models were well tolerated by ¹⁷⁷Lu-PCTA-cetuximab treatment, and no apparent body weight loss was observed, suggesting that the 12.95 MBq dose used in this study had no observable toxicity on mice.

### EXAMPLE 3

### Small Animal PET Imaging of ⁶⁴Cu-PCTA-Cetuximab

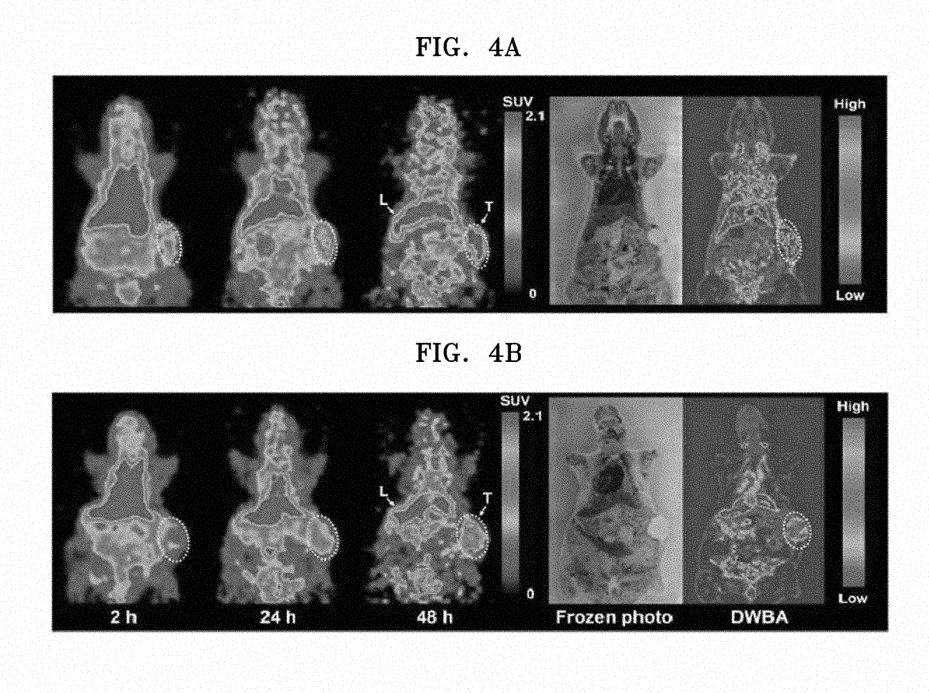

Small-animal PET imaging was performed to evaluate the potential of ⁶⁴Cu-PCTA-cetuximab as an immuno-PET imaging agent for EGFR expression level in SNU-1066 tumor-bearing mice, and the results are shown in FIGS. 4A and 4B.

⁶⁴Cu-PCTA-cetuximab (3.7 MBq) was intravenously injected into the mice, and static scans were acquired for 60 min at 2 hrs, 24 hrs, 48 hrs, and 72 hrs after injection using a small animal PET scanner (microPET R4; Concorde Microsystems, Knoxville, Tenn., USA). To evaluate the specificity of EGFR-expressing tumor targeting of ⁶⁴Cu-PCTA-cetuximab, excess cold cetuximab (2 mg/head) was intravenously injected for blocking experiments. Quantitative data were expressed as standard uptake value (SUV). Images were visualized using ASIPro display software.

FIGS. 4A and 4B show small animal PET imaging of ⁶⁴Cu-PCTA-cetuximab in SNU-1066 HNSCC xenograft models.

FIG. 4A shows ⁶⁴Cu-PCTA-cetuximab selectively localized in SNU-1066 tumors. After PET imaging, mice were immediately euthanized and frozen, and frozen section photo and digital whole body autoradiography (DWBA) images were obtained. DWBA images showed a similar distribution pattern with the PET images.

FIG. 4B shows that in blocking experiments, ⁶⁴Cu-PCTA-cetuximab uptake in tumor was markedly reduced by pre-treated cold excess cetuximab. Tumors are indicated by white dotted lines. T, SNU-1066 tumor; L, liver.

PET images were acquired at 2 hrs, 24 hrs, and 48 hrs after injection of ⁶⁴Cu-PCTA-cetuximab, and represented as SUV. SNU-1066 tumors were clearly visualized on PET images and the tumor uptake of ⁶⁴Cu-PCTA-cetuximab peaked at 48 hrs post-injection. Physiological liver uptake was also observed, but gradually reduced over time. The tumor SUV of ⁶⁴Cu-PCTA-cetuximab was 0.9±0.2, 1.9±0.3 and 3.0±0.7 at 2 hrs, 24 hrs, and 48 hrs post-injection, respectively. Immuno-PET images were well consistent with the biodistribution data.

Blocking experiment with excess dose of cetuximab resulted in 56.7% reduced tumor uptake of ⁶⁴Cu-PCTA-cetuximab, indicating the EGFR targeting specificity of ⁶⁴Cu-PCTA-cetuximab.

While the present disclosure has been described with reference to preferred embodiments, it will be understood by those skilled in the art that the present disclosure may be implemented in a different specific form without departing from essential characteristics thereof. Therefore, it should be understood that embodiments described herein should be considered in a descriptive sense only and not for purposes of limitation. The scope of the present disclosure is defined by the appended claims rather than by the foregoing description, and all differences within the scope of equivalents thereof should be construed as being included in the present disclosure.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A conjugate of the following formula 1:
[X]-R-epidermal growth factor receptor (EGFR) inhibitor [Formula 1]
(wherein X is a radioisotope and R is a ligand).

2. The conjugate of claim 1, wherein the radioisotope (X) is any one selected from the group consisting of lutetium-177 (¹⁷⁷Lu), copper-64 (⁶⁴Cu), copper-67 (⁶⁷Cu), gallium-68 (⁶⁸Ga), zirconium-89 (⁸⁹Zr), yttrium-86 (⁸⁶Y), yttrium-90 (⁹⁰Y), technetium-99m (^{99m}Tc), indium-111 (¹¹¹In), promethium-149 (¹⁴⁹Pm), scandium-47 (⁴⁷Sc), rhodium-105 (¹⁰⁵Rh), tin-117m1 (^{117m1}Sn), tin-117m2 (^{117m2}Sn), terbium-161 (¹⁶¹Tb) and a combination thereof.

3. The conjugate of claim 1, wherein the EGFR inhibitor is any one selected from the group consisting of cetuximab, panitumumab, gefitinib, erlotinib, icotinib, lapatinib, neratinib, apatinib, ceritinib, dacomitinib, and canertinib.

4. The conjugate of claim 1, wherein the ligand (R) is a metal chelate ligand that is conjugated to the EGFR inhibitor and forms a complex with the radioisotope.

5. The conjugate of claim 1, wherein the ligand (R) is any one selected from the group consisting of 3,6,9,15-tetraazabicyclo[9.3.1]-pentadeca-1(15),11,13-triene-3,6,9,-triacetic acid (PCTA), 1,4,7,10-tetraazacyclododecane-1,4,7,10-tetraacetic acid (DOTA), diethylene triamine penta acetic acid (DTPA), 1,4,7,10-tetraazacyclododecane-1,4,7-triacetic acid (DO3A), 1,4,7-triazacyclononane-1,4,7-triacetic acid (NOTA), 1,4,7-triazacyclononane, 1-glutaric acid-4,7-acetic acid (NODAGA), 1,4,8,11-tetraazacyclotetradecane-N,N′,N″,N′″-tetraacetic acid (TETA), 1,4,8,11-tetraazacyclotetradecane-1,4,8-triacetic acid (TE3A), 1,4,8,11-tetraaza bicyclo hexadecane-4,11-diacetic acid (TE2A), cyclen, cyclam, and desferrioxamine (DFO or DFOA).

6. The conjugate of claim 1, wherein the radioisotope (X) is any one selected from lutetium-177 (¹⁷⁷Lu), copper-64 (⁶⁴Cu), and a combination thereof.

7. The conjugate of claim 1, wherein the EGFR inhibitor is cetuximab.

8. The conjugate of claim 1, wherein the ligand (R) is PCTA.

9. The conjugate of claim 1, wherein the EGFR inhibitor is cetuximab, the radioisotope (X) is lutetium-177 (¹⁷⁷Lu) or copper-64 (⁶⁴Cu), and the ligand (R) is a metal chelate ligand that forms a complex with [¹⁷⁷Lu] or [⁶⁴Cu] and is conjugated to cetuximab.

10. The conjugate of claim 1, wherein the conjugate is imaged by any one imaging device selected from the group consisting of positron emission tomography (PET), single-photon emission computed tomography (SPECT), magnetic resonance imaging (MRI), computed tomography (CT), an optical imaging technique, an ultrasound (US) technique, Cherenkov luminescence imaging, and a combination thereof.

11. A method for tumor treatment and diagnosis by targeted drug delivery, wherein the conjugate of the following formula 1 of claim 1 is used:
[X]-R-epidermal growth factor receptor (EGFR) inhibitor [Formula 1]
(wherein X is a radioisotope and R is a ligand)

12. The method of claim 11, wherein a tumor treated and diagnosed by the method is head and neck cancer.

13. The method of claim 12, wherein the tumor is head and neck cancer resistant to EGFR inhibitors.

14. The method of claim 11, wherein the EGFR inhibitor is cetuximab and a tumor treated and diagnosed by the method is a tumor resistant to cetuximab.

15. The method of claim 11, wherein the ligand (R) is 3,6,9,15-tetraazabicyclo[9.3.1]-pentadeca-1(15),11,13-triene-3,6,9,-triacetic acid (PCTA).

16. The method of claim 11, wherein the radioisotope (X) is any one selected from lutetium-177 (¹⁷⁷Lu), copper-64 (⁶⁴Cu), and a combination thereof.

17. A method of preparing the conjugate of claim 1, the method comprising conjugating the ligand (R) to the EGFR inhibitor; and labeling the EGFR inhibitor-conjugated ligand (R) with the radioisotope (X).
